(19) United States
(12) Patent Application Publication
(10) Pub. No.: US 2017/0119468 A1
(43) Pub. Date: May 4, 2017

(54) MICROSURGICAL INSTRUMENT

(71) Applicant: Peregrine Surgical, Ltd., New Britain, PA (US)

(72) Inventor: Theodore Todd Richmond, Doylestown, PA (US)

(73) Assignee: Peregrine Surgical, Ltd., New Britain, PA (US)

(21) Appl. No.: 15/337,412

(22) Filed: Oct. 28, 2016

Related U.S. Application Data
(60) Provisional application No. 62/248,676, filed on Oct. 30, 2015.
(60) Provisional application No. 62/408,278, filed on Oct. 14, 2016.

(51) Int. Cl. A61B 18/22; A61F 9/008

(52) U.S. Cl. CPC A61B 18/22; A61F 9/008; A61B 2018/2005

(57) ABSTRACT

A microsurgical instrument including a handpiece defining a handpiece bore is provided. The instrument includes a first needle arranged at least partially within the handpiece bore, and an optical fiber extending through the handpiece bore and fixed to an interior of the first needle. The handpiece is rotatable relative to the optical fiber and the first needle.

### INCORPORATION BY REFERENCE

The following documents are incorporated herein by reference as if fully set forth: U.S. Provisional Application No. 62/248,676, filed Oct. 30, 2015; and U.S. Provisional Application No. 62/408,278, filed Oct. 14, 2016.

### FIELD OF INVENTION

The present invention relates generally to medical devices, and more particularly to a microsurgical instrument's handpiece.

### BACKGROUND

In ophthalmic surgery, adequate visualization of interior portions of the eye is critical to the success of the surgical procedure. The development of endoillumination has greatly improved the way surgeons are able to visualize the interior portions of the eye. Most common ophthalmic surgery procedures involve making three stab incisions (i.e., sclerotomy) for accessing the eye through the vitreous chamber. One of these incisions is used for insertion of the illuminator. A second incision is ultimately used for insertion of an infusion cannula, which is used to introduce fluids to prevent collapse and otherwise maintain the integrity of the eye. A third incision is made in the eye for insertion of the specific surgical instruments to be used for performing the surgery.

There are various types of illuminators employed in ophthalmic surgery. These illuminators typically employ an optical fiber having a flexible elongate length with opposed proximal and distal ends. The optical fiber is usually encased in an elongate tubular jacket with some form of cladding. The proximal end of the optical fiber is secured to a connector adapted for coupling to a corresponding illumination light source for supplying the illumination light through the optical fiber. The distal end of the optical fiber is inserted through an incision in the eye and the illumination light emitted therefrom is dispersed throughout the vitreous chamber of the eye.

Existing microsurgical instruments typically include an optical fiber, a jacket encompassing a majority of the optical fiber, a handpiece including a bore through which the optical fiber extends, and a needle attached to the handpiece through which guides the fiber to the incision site. These existing microsurgical instruments are typically designed such that the fiber, the jacket, and the needle are bonded to the handpiece by epoxy or other bonding material. Due to the integral connection between each of these components of the microsurgical instrument, motion experienced by any one of the components is translated to each of the remaining components, resulting in residual motion in the form of recoil or vibrations. This residual motion is undesirable due to the precise nature of surgery, particularly ophthalmic surgery.

Existing microsurgical instruments can also include an optical fiber with a tapered end that promotes a wide angle effect to spread light evenly throughout a patient's eye. These tapered optical fibers are helpful for providing wider illumination of a patient's eye as compared to a flat-ended optical fiber. However, since the tapered fiber is at least partially exposed and extends beyond the end of handpiece needle or tube, the fiber creates a glare for the surgeon, which is undesirable.

Accordingly, there is a need for a microsurgical instrument that reduces residual motion between sub-components and simplifies construction of the handpiece, while also shielding glare from a wide angle optical fiber probe.

### SUMMARY

A microsurgical instrument is provided that includes a free-rotating handpiece with respect to an optical fiber extending through the handpiece. The handpiece includes a handpiece bore and at least one first needle arranged at least partially within the handpiece bore. An optical fiber extends through the handpiece bore, and is fixed to an interior of the at least one first needle. The optical fiber and the at least one first needle are rotatable relative to the handpiece such that the handpiece is free-rotating with respect to the optical fiber.

### BRIEF DESCRIPTION OF THE DRAWINGS

The following drawings are illustrative of preferred embodiments of the present invention, and are not intended to limit the invention as encompassed by the claims forming part of the application, wherein like items are identified by the same reference designations:

FIG. 1 is an exploded view of an optical fiber, ring, and needle set components of a microsurgical instrument according to a first embodiment of the present invention.

FIG. 2 is a cross-sectional view of the assembled microsurgical instrument, including the components shown in FIG. 1.

FIG. 3 is a cross-sectional view of at least one needle and a disc component of a microsurgical instrument according to a second embodiment of the present invention.

FIG. 4 is a cross-sectional view of a handpiece of the second embodiment of the microsurgical instrument, including the components shown in FIG. 3.

FIG. 5 is a view of a partially assembled tapered optical fiber, jacket, and needle.

FIG. 6 is a cross-sectional view of an assembled microsurgical instrument including a tapered optical fiber.

FIG. 7 is a magnified view of a tapered optical fiber.

### DETAILED DESCRIPTION OF THE PREFERRED EMBODIMENTS

A microsurgical instrument is disclosed that includes a free-rotating handpiece relative to an optical fiber extending through the handpiece. The optical fiber is axially secured within the handpiece by a needle. The term “needle” is understood to mean a tube-shaped sleeve. The optical fiber extends through the needle and is fixed to an interior of the needle. The needle is axially captively secured between a ring arranged within the handpiece and a shoulder defined by the handpiece. The needle is free to rotate within the handpiece, and the optical fiber is also free to rotate within the handpiece.

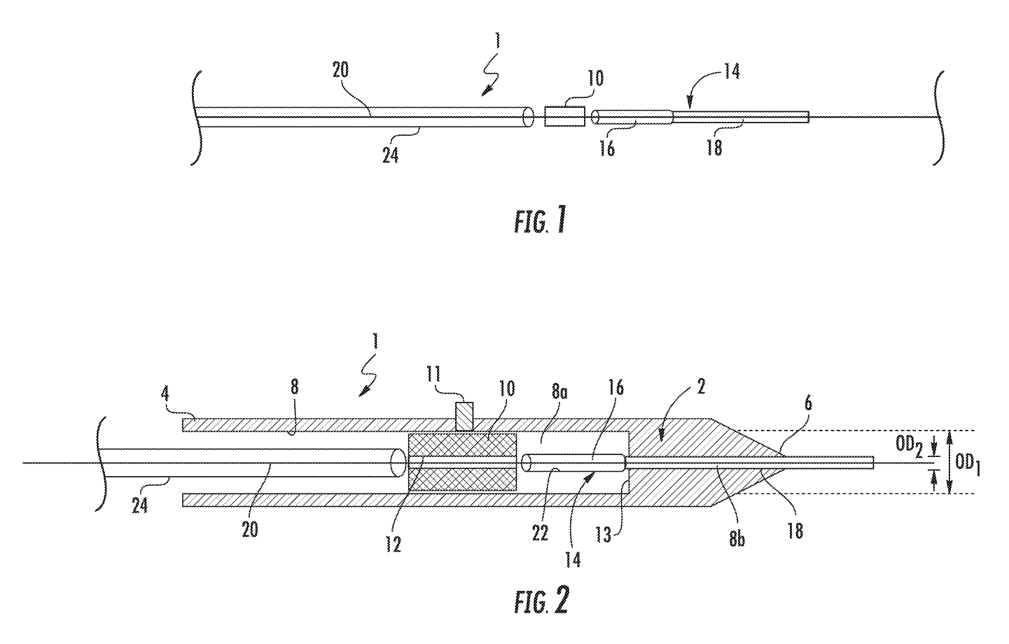

A first embodiment of a microsurgical instrument 1 is shown in FIGS. 1 and 2. The microsurgical instrument 1 includes a handpiece 2 having a first axial end 4 and a second axial end 6. A handpiece bore 8 extends between the first axial end 4 and the second axial end 6. The handpiece bore 8 preferably includes a stepped configuration having a retention shoulder 13 defined between a first bore section 8a and a second bore section 8b. An outer diameter OD₁ of the first bore section 8a is greater than an outer diameter OD₂ of the second bore section 8b. A ring 10 is fixedly arranged within the handpiece bore 8 and the ring 10 includes a ring bore 12. The ring 10 is preferably fixedly arranged within the handpiece bore 8 via a fastening element 11. As shown in FIG. 2, the fastening element 11 is more preferably a screw. One of ordinary skill in the art would recognize from the present disclosure that any type of fastening can be used to fasten the ring 10 to the handpiece 2, including without limitation, a bonding epoxy or an interference or friction fit.

The microsurgical instrument 1 includes a needle set 14 having a first needle 16 and a second needle 18. One of ordinary skill in the art would recognize from the present application that any number of needles and configurations could be used for the needle set 14. The first needle 16 is captively secured in the handpiece bore 8 between the ring 10 and the second axial end 6 of the handpiece 2. The second needle 18 is coaxially arranged within and fixed to the first needle 16. The first needle 16 and the second needle 18 are preferably fixed to each other via a bonding epoxy. One of ordinary skill in the art would recognize that the first needle 16 and the second needle 18 can be fixed to each other via a variety of fastening configurations. The first needle 16 is rotatable within the handpiece bore 8 and the second needle 18 rotates in unison with the first needle 16. As shown in FIG. 2, the first needle 16 is preferably captively secured in the first bore section 8a of the handpiece bore 8 between the retention shoulder 13 and the ring 10. The second needle 18 preferably extends through the second bore section 8b of the handpiece bore 8b.

An optical fiber 20 extends through the handpiece bore 8 and the optical fiber 20 is fixed to an interior 22 of at least one of the first needle 16 or the second needle 18. The optical fiber 20 is rotatable relative to the handpiece 2 and the ring 12. By allowing the optical fiber 20 to rotate with respect to the handpiece 2, and allowing the first and second needles 16 and 18 to rotate relative to the handpiece 2, any recoil or motion experienced by the handpiece are dampered or localized. In other words, any unwanted motion or vibration is significantly “localized” to a specific component of the microsurgical instrument 1.

As shown in FIG. 2, a jacket 24 is preferably arranged at least partially within the first axial end 4 of the handpiece 2. The optical fiber 20 is at least partially coaxially arranged within the jacket 24. In one embodiment, the optical fiber 20 and the jacket 24 are fastened to each other via epoxy in a separate connector component which is not shown in the drawings.

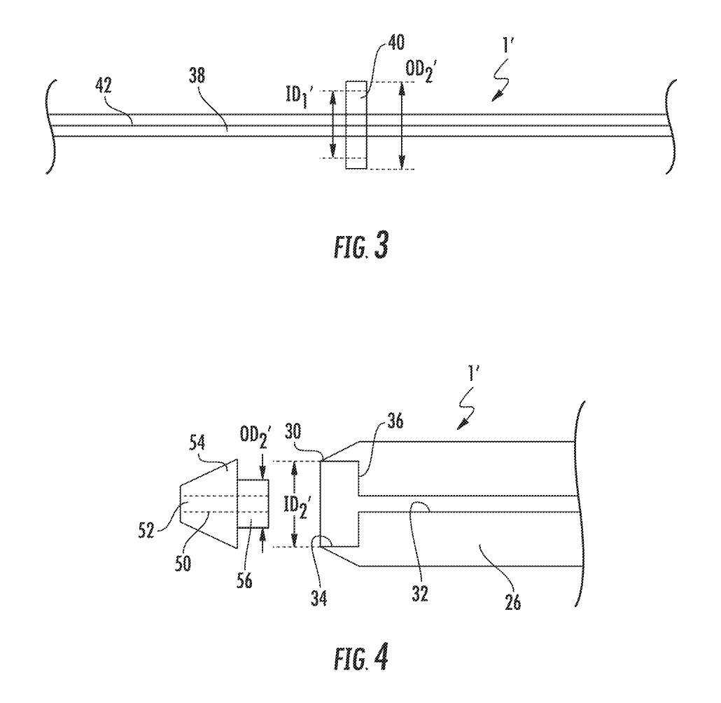

A second embodiment of the microsurgical instrument 1′ shown in FIGS. 3 and 4. Similar to the first embodiment, the microsurgical instrument 1′ of the second embodiment includes a handpiece 26 having a first axial end (not shown) and a second axial end 30. A handpiece bore 32 extends from the first axial end to the second axial end 30. The handpiece bore 32 includes an enlarged opening 34 with a shoulder 36 having a first inner diameter ID₁′ at the second axial end 30 of the handpiece 26. At least one needle 38 is arranged within the handpiece 26, and a disc 40 is coaxially fixed to the at least one needle 38. The disc 40 includes an opening having inner diameter ID₂′. The disc 40 has a first outer diameter OD₁′ and the first outer diameter OD₁′ is less than the first inner diameter ID₁′ of the shoulder 36. An optical fiber 42 extends through the handpiece bore 32 and is at least partially coaxially arranged within the at least one needle 38. Once mounted, the optical fiber 42 and the at least one needle 38 are rotatable with respect to the handpiece 26.

A nosepiece 50 includes a nosepiece bore 52, and the nosepiece 50 is configured to be retained within the enlarged opening 34 of the handpiece 26 preferably via a press fit. However, one of skill in the art would recognize that other means can be used to retain the nosepiece 50 to the handpiece 26. The nosepiece 50 includes an enlarged head 54 preferably having a frusto-conical profile, and a base portion 56 preferably having a cylindrical profile. In one embodiment, the inner diameter ID₂′ of the disc 40 is greater than the outer diameter OD₂′ of the base portion 56. Once assembled, the nosepiece 50 is securely retained in the enlarged opening 34 of the handpiece 26 and acts as a stopper against the disc 40, which is captively retained between the shoulder 36 of the handpiece 26 and the nosepiece 50. The disc 40 is captively retained such that the disc 40 is rotatable within the handpiece, and therefore the at least one needle 38 and the optical fiber 42 are rotatable within the handpiece 26.

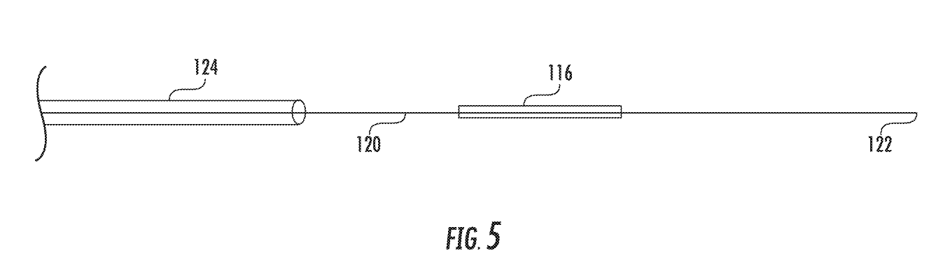

A third embodiment of a microsurgical instrument 101 is shown in FIGS. 5-7. The microsurgical instrument 101 includes an optical fiber 120 with a tapered end 122. As shown in FIG. 7, the tapered end 122 has a frusto-conical profile. The profile of the tapered end 122 provides wider illumination compared to an approximate 40° of illumination of a flat-ended fiber. In one embodiment, the tapered end 122 provides wide illumination that is at least 2.5 times greater than the degree of illumination provided by a flat-ended fiber. In another embodiment, the tapered end 122 provides at least 90° of illumination. In another embodiment, the tapered end 122 provides approximately 105° of illumination.

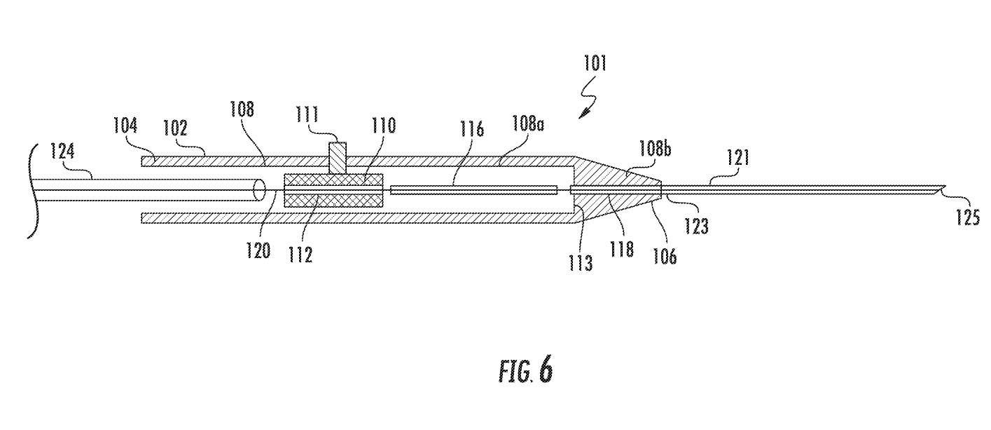

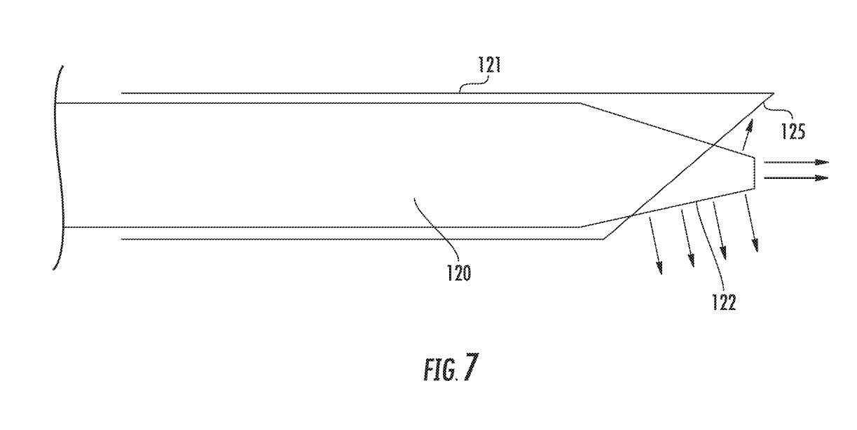

The optical fiber 120 is surrounded by a jacket 124, similar to the first embodiment of the microsurgical instrument 1. In an assembled view shown in FIG. 6, the optical fiber 120 is arranged within a handpiece 102. The handpiece 102 is similar to the first embodiment of the handpiece 2 described above, and similarly includes a ring 110. The ring 110 is preferably fixedly arranged within a handpiece bore 108 of the handpiece 102 by a fastener 111. In one embodiment, the fastener 111 is a screw. One of ordinary skill in the art would recognize from the present disclosure that any type of fastener can be used to fasten the ring 110 to the handpiece 102, including without limitation, a bonding epoxy, an interference fit, or a friction fit. The ring 110 includes a ring bore 112 that defines a passage for the optical fiber 120. The handpiece bore 108 extends between a first axial end 104 and a second axial end 106 of the handpiece 102. The handpiece bore 108 preferably includes a stepped configuration defining a retention shoulder 113 at a transition between a first bore section 108a and a second bore section 108b. Similar to the first embodiment, the first bore section 108a is wider than the second bore section 108b.

The third embodiment of the microsurgical instrument 101 includes a first needle 116 and a second needle 118. As shown in FIG. 6, the first needle 116 is captively secured within the handpiece bore 108 between the ring 110 and the second axial end 106 of the handpiece 102. The second needle 118 is arranged at least partially within the second bore section 108b at the second axial end 106 of the handpiece 102. As shown in FIG. 6, the first needle 116 and the second needle 118 are separately formed and are spaced apart from each other. The first needle 116 defines a passage for the optical fiber 120, and the optical fiber 120 is fixed to an interior of the first needle 116. The second needle 118 is fixed within the second bore section 108b of the handpiece 102.

A shielding tube 121 partially overlaps in an axial direction with the second needle 118 and extends away from the second axial end 106 of the handpiece 102. In one embodiment, the second needle 118 and the shielding tube 121 are arranged coaxial with each other. In another embodiment, the second needle 118 and the shielding tube 121 are integrally formed and a single needle/shielding tube extends from the handpiece 102. As shown in FIGS. 6 and 7, the optical fiber 120 extends within the shielding tube 121. A first end 123 of the shielding tube 121 is arranged partially within the second axial end 106 of the handpiece 102 and the shielding tube 121 is rotationally fixed with the handpiece 102. A second end 125 of the shielding tube 121 includes a beveled edge, shown most clearly in FIG. 7. The beveled edge of the shielding tube 121 provides an angled shield that blocks a portion of the light (shown schematically in FIG. 7) emitted from the tapered end 122 of the optical fiber 120. The shielding tube 121 at least partially overlaps with the tapered end 122 of the optical fiber 120. In a preferred embodiment, the shielding tube 121 completely overlaps with the tapered end 122 of the optical fiber 120, i.e. the shielding tube 121 extends beyond the tip of the optical fiber 120. Although the beveled edge of the shielding tube 121 is illustrated as an angled straight edge in the drawings, one of ordinary skill in the art would recognize that other shapes could be used to block light from the optical fiber 120. The shielding tube 121 is fixed to the handpiece 102 such that a surgeon can grip the handpiece 102 and rotate the handpiece 102 to adjust an angle of the shielding tube 121 relative to the optical fiber 120. The handpiece 102 can be rotated such that the shielding tube 121 is arranged in a line of sight between the tapered end 122 of the optical fiber 120 and a surgeon's eyes. Due to the free-rotating arrangement of the handpiece 102 relative to the optical fiber 120, rotation of the handpiece 102 (in order to adjust a position of the shielding tube 121) does not alter a position of the optical fiber 120. Based on this free-rotating arrangement, there is no undesirable torque or snap-back forces on the optical fiber 120 and the jacket 124 during positioning of the handpiece 102 and the shielding tube 121. Additionally, the surgeon can manipulate the handpiece 102 and continuously rotate the handpiece 102 with the shielding tube 121 such that the surgeon continuously blocks any glare from the tapered end 122 of the optical fiber 120.

The forgoing discussion discloses and describes merely exemplary embodiments of the present invention. One skilled in the art will readily recognize from such discussion, and from the accompanying claims, that various changes, modifications, and variations can be made therein without departing from the spirit and scope of the invention as defined in the following claims.

What is claimed is:

1. A microsurgical instrument comprising:
a handpiece defining a handpiece bore;
a first needle arranged at least partially within the handpiece bore; and
an optical fiber extending through the handpiece bore, and fixed to an interior of the first needle;
wherein the handpiece is rotatable relative to the optical fiber and the first needle.

2. The microsurgical instrument of claim 1, further comprising a shielding tube fixed to an axial end of the handpiece.

3. The microsurgical instrument of claim 2, wherein the shielding tube includes a beveled edge and the optical fiber terminates within the beveled end of the shielding tube.

4. The microsurgical instrument of claim 1, wherein the optical fiber includes a tapered end.

5. The microsurgical instrument of claim 1, further comprising a ring axially fixed within the handpiece bore, the ring including a ring bore through which the optical fiber extends, and the first needle is rotatable within the handpiece bore.

6. The microsurgical instrument of claim 5, wherein the first needle is captively secured in an axial direction between the ring and an internal shoulder of the handpiece.

7. A microsurgical instrument comprising:
a handpiece defining a handpiece bore;
an optical fiber extending through the handpiece bore, the optical fiber includes a tapered end;
a ring axially fixed within the handpiece bore and defining a ring bore, the optical fiber extending through the ring bore;
a first needle arranged within the handpiece bore, the optical fiber is fixed to an interior of the first needle, and the first needle is captively axially secured between the ring and an internal shoulder defined by the handpiece bore;
a jacket arranged at least partially within a first axial end of the handpiece, and the optical fiber is arranged coaxially within the jacket; and
a shielding tube fixed to a second axial end of the handpiece, a first end of the shielding tube is fixed to the handpiece and a second end of the shielding tube includes a beveled edge, and the tapered end of the optical fiber is arranged within the beveled edge of the shielding tube;
wherein the shielding tube is rotationally fixed to the handpiece, and the handpiece is rotatable relative to the optical fiber and the first needle.

8. The microsurgical instrument of claim 7, wherein the optical fiber is fixed to the interior of the first needle by an epoxy.

9. The microsurgical instrument of claim 7, wherein the beveled edge of the shielding tube is formed as an angled straight edge.

10. The microsurgical instrument of claim 7, wherein the first end of the shielding tube is glued to an interior of the handpiece.

11. The microsurgical instrument of claim 7, wherein the tapered end of the optical fiber has a frusto-conical profile.

12. The microsurgical instrument of claim 7, wherein the tapered end of the optical fiber provides at least 90° of illumination.

13. The microsurgical instrument of claim 7, wherein the tapered end of the optical fiber provides 105° of illumination.
